(19) United States
(12) Patent Application Publication
(10) Pub. No.: US 2022/0229056 A1
(43) Pub. Date: Jul. 21, 2022

(54) BREATHALYZER AND SENSOR FOR DETECTING HALITOSIS AND GENERAL HEALTH ISSUES

(71) Applicant: ORTHONU, LLC, Rumson, NJ (US)

(72) Inventor: Sima Yakoby Epstein, Rumson, NJ (US)

(21) Appl. No.: 17/557,695

(22) Filed: Dec. 21, 2021

Related U.S. Application Data
(60) Provisional application No. 63/140,247, filed on Jan. 21, 2021.

(51) Int. Cl. G01N 33/569; G01N 33/543; G01N 1/22

(52) U.S. Cl. CPC G01N 33/56911; G01N 33/54388; G01N 2001/2244; G01N 2800/18; G01N 1/2214

(57) ABSTRACT

A saliva sensing device for detecting oral health and/or overall health includes a chemically treated test strip biomarker targeted to identify bacteria in the saliva. A breathalyzer for detecting bacteria in the mouth of a user includes a housing, a sensor, and an analyzer. The sensor is coupled to the housing and extends outwardly therefrom for placing in the mouth of user and for collecting a saliva sample. The analyzer is for testing saliva that contacts the sensor.

### CROSS-REFERENCE TO RELATED APPLICATION

This application claims priority to U.S. Provisional Patent Application No. 63/140,247, filed Jan. 21, 2021, the disclosure of which is incorporated herein by reference in its entirety.

### FIELD

The invention concerns a breathalyzer and sensor for detecting halitosis and general health issues. In particular, the invention concerns a device for sensing health-related attributes from saliva and/or breath.

### BACKGROUND

In orthodontic care of patients, there are at least three areas of concern that must be addressed, and which are currently under-addressed in consumer solution products. These include 1) emergency care, 2) oral hygiene, and 3) visual challenges. Emergency care is needed to address discomfort, pain, sensitivity, ulcerations, dry mouth, and the like. Oral hygiene involves long term oral care and prevention. Oral hygiene includes maintenance care for preventing halitosis and preventing or treating gingivitis and the increase in gram negative bacteria on the tongue and periodontium that are associated with orthodontia care. Avoiding gingivitis and reducing gram negative bacteria in a patient's mouth can prevent and/or deter bone loss and inflammation. This can help to maintain a healthy mouth, tongue, gums, and fresh breath. Visual challenges include the appearance of white decalcifications on teeth due to the start of cavities that can be attributed to an increase of gram-positive bacteria, among other challenges.

Gram negative bacteria lives on the tongue and inside the gum line around the periodontium. Gram negative bacteria is a main cause of halitosis in the mouth. Gram positive bacteria is found in the plaque accumulation of the pellicle that forms on the enamel of the tooth that then starts the decalcification process that can lead to cavities, also causing halitosis. Targeting these two types of bacteria with different ingredients and tools and differentiating them can solve many issues inherent in the changes in microbiome during orthodontic treatment.

In addition, overall health has been tied to health of the teeth and oral cavity. Oral health can offer clues as to overall health. Problems in the mouth can affect the rest of the body. The mouth normally teems with bacteria, most of which is harmless. The mouth is an entry point to the digestive and respiratory tracts, and some of the bacteria present in the mouth can cause disease. Normally the body's natural defenses a good oral health care, such as daily brushing and flossing, can keep bacteria under control. Without proper oral hygiene, bacterial can lead to oral infections, such as tooth decay and gum disease.

Saliva is an important component in the mouth because it washes away food and neutralizes acids produced by bacteria in the mouth. Saliva helps to protect against microbes that multiply and lead to disease. Certain common medications, such as decongestants, antihistamines, painkillers, diuretics and antidepressants, can reduce saliva flow.

Oral health can contribute to various diseases, such as endocarditis, cardiovascular disease, pregnancy and birth complications, and pneumonia, among others. For example, endocarditis, an infection of the inner lining of the hear chambers or valves, typically occurs when bacteria from another part of the body, such as the mouth, spreads through the bloodstream and attaches to certain areas in the heart. Some research suggests that heart disease, clogged arteries and stroke might be linked to the inflammation and infections that oral bacterial can cause. Periodontitis has been linked to premature birth and low birth weight. Certain bacteria in the mouth can be pulled into the lungs, which can cause pneumonia and other respiratory diseases.

Certain conditions may also affect overall oral health, such as diabetes, HIV/AIDS, osteoporosis, Alzheimer's disease, among others. Diabetes, for example, puts gums at greater risk by reducing the body's resistance to infection. Research shows that people with gum disease have a harder time controlling blood sugar levels. Oral problems, such as lesions are common with people having HIV/AIDS. Osteoporosis can cause periodontal bone loss and tooth loss. Worsening oral health is often seen in Alzheimer's disease progression.

Thus, oral health is important to overall health. Because of the lack of home care solutions, patients often simply live with the bad breath and plaque that builds up on their teeth. Patients are often unaware that their oral health has an impact on their overall health. On-the-go solutions and home-care solutions would assist patients in determining whether they need treatment to improve oral care.

There is also a need for an easy system for determining overall health based upon oral health. In particular, it is desirable to link gum and mouth health to other diseases by testing saliva. Such a system would permit a person to test their overall health at home.

### SUMMARY

A saliva sensing device for detecting oral health and/or overall health includes a chemically treated test strip biomarker targeted to identify bacteria in the saliva. The test strip may be targeted to analyze one or more VOCs, wherein the VOCs include one or more of ketones, alcohols, aldehyde, and sulfur. The test strip may measure unbound hormones in the saliva, with the measured unbound hormones being tied to one or more disease states. The test strip may measure lipidomic biomarkers. The test strip measures key lipids, and the measured key lipids are used in diagnosing diseases, diagnosing disease states, and developing treatment plans. The test strip may be targeted to test pathogens including one or more of S. mutans, S. sanguis, P. gingivalis, and F. nucleatum. The test strip may have a chemical treatment to detect gram negative bacteria. The test strip may have a chemical treatment to detect gram positive bacteria. The test strip may have a chemical treatment to detect one or more of the following:

1) Bacteria located in saliva due to low PH of saliva;

2) Bacteria located in saliva due to acidic saliva;

3) Bacteria production caused by insufficient hydration;

4) Bacteria production caused by insufficient salivary flow; and

5) Bacteria due to food related oral hygiene.

A breathalyzer for detecting bacteria in the mouth of a user includes a housing, a sensor coupled to the housing and extending outwardly therefrom for placing in the mouth of user and for collecting a saliva sample, and an analyzer for testing saliva that contacts the sensor. The sensor may include a test strip for collecting saliva. The test strip may be a chemically treated cloth biomarker. The chemically treated cloth biomarker may be configured to identify bacteria in saliva that contacts the cloth test strip. The test strip may detect the level of bacteria present in the saliva and the level of bacteria may be determined based upon a color displayed on the test strip. The test strip may detect the level of bacteria present in the saliva and the level of bacteria may be determined based upon a color intensity displayed on the test strip. The test strip may measure unbound hormones in the saliva, with the measured unbound hormones being tied to disease states. The test strip may measure lipidomic biomarkers. The test strip may measure key lipids, and the measured key lipids are used in diagnosing diseases, diagnosing disease states, and developing treatment plans. The test strip may be targeted to a specific disease state. The disease state may include one or more of diabetes, heart disease, autoimmune disorders, oral cancer, Alzheimer's, and gastrointestinal diseases.

### BRIEF DESCRIPTION OF THE DRAWINGS

FIG. 1 depicts test strips for use in detecting oral health according to the invention according to the invention;

FIG. 2 depicts a test strip holder;

FIG. 3 depicts a test strip holder with a test strip positioned in the holder;

FIG. 4 depicts a test strip holder with a test strip positioned in the holder;

FIG. 5 depicts a test strip holder having a cover; and

FIG. 6 depicts a front view of a breathalyzer/detector/analyzer according to the invention for testing breath and/or saliva.

### DETAILED DESCRIPTION

Oral health and hygiene are important for health. Poor oral health can result in disease to the body. Poor oral health can also be predictive of disease and overall health. The present invention is designed to test oral health to determine if a person needs to improve their oral hygiene and to assist in disease diagnosis, among other things.

Oral health is particularly important in the context of orthodontia treatment. During orthodontia treatment, patients are often challenged with oral hygiene. Food can get stuck between teeth and, as teeth shift, food can get lodged in the gums. This can cause swollen gums and excessive bacteria build up on the tongue and mouth. Patients are more likely to have bad breath from excess bacteria. A breathalyzer 10 according to the invention provides a mechanism to measure the bacteria level in a patient's mouth and saliva to understand if a patient has bacteria present that may cause bad breath. Specifically, the breathalyzer 10 can determine which type of bacteria is present so that it can be properly treated.

The mouth, including mouth bacteria, is the gateway of these negative agents into the body. Saliva may be used to measure diagnostics amongst clinicians due to its measurable amount of free, unbound hormones that can be used to diagnose irregular hormone levels. In addition, hundreds of diverse types of bacteria live in the oral cavity, they are distinct to the palate, teeth, tongue, and tonsillar area.

Discerning these oral floras can illuminate bacterial infections that are present in the mouth and eventually continue into the human body. These can be connected to systemic infections, preliminary signs of bacterial endocarditis, pneumonia, cardiovascular disease, diabetes, gastrointestinal disorders and even Alzheimer's. The goal is to identify the exact bacteria that is related to each medical diagnosis of a systemic infection, precursor or symptom of a disease or disorder. Early diagnosis can be the difference between finding a solution and waiting for the symptoms to create a challenge. Thus, the present invention, as described herein, may serve as an early detection tool. The present invention connects the oral microbiota with the medical world and allows all specialists to be on the cutting edge of early detection and prevention.

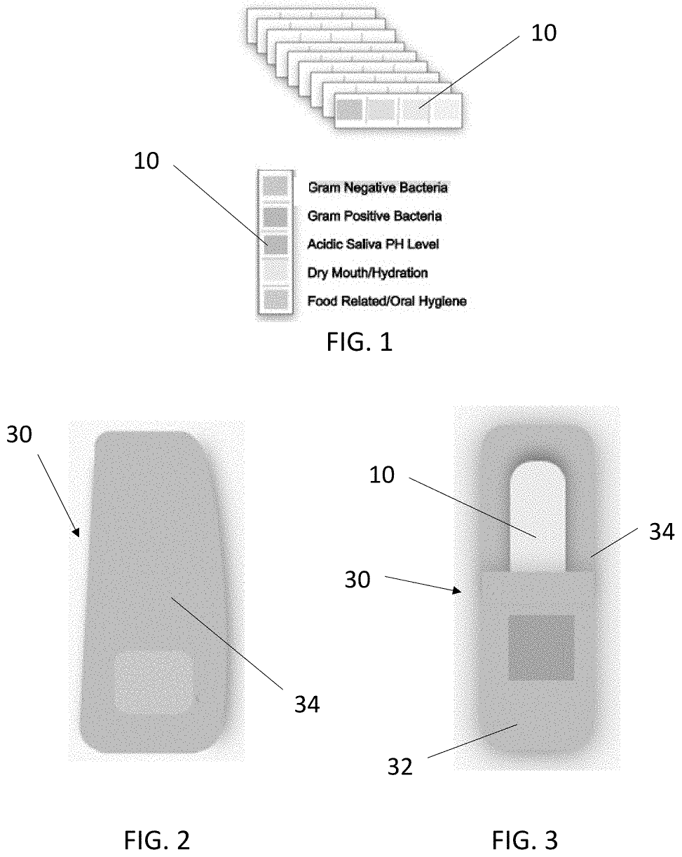

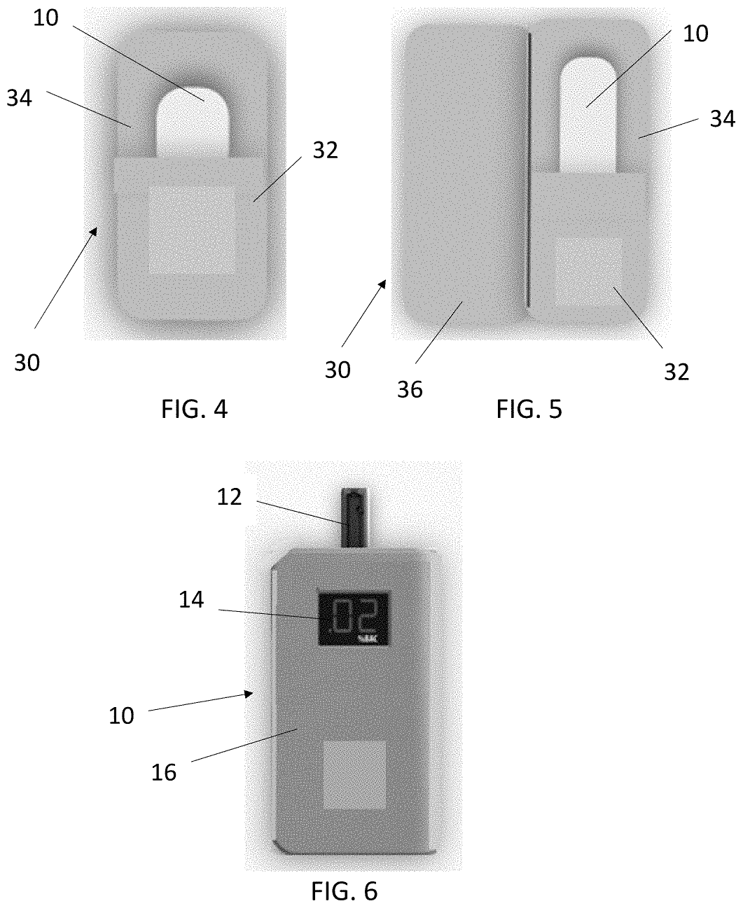

As shown in FIGS. 1-5, a test strip 20 is provided. The test strip 20 may be used with a breathalyzer 10 or testing device or may be used by itself. The test strip 20 is a small, pre-treated strip 20 that can measure the level of Ph, acidity, and bacteria, among other things, on the patient's tongue or in saliva. The test strip 20 may be considered a medical strip and is positioned on the tongue for approximately 10-15 seconds, until it is saturated, to allow the reaction. This is an easy-to-use tool that can show levels of bacteria and other levels of measured variables. As shown in FIGS. 2-5, the test strips 20 may be positioned in a holder 30, such as a case 30. The case may have a front 32 and a back 34, with the front 32 presenting a half wall and the strips 20 can be positioned between the front half wall 32 and the back 34. The holder 30 may also have a cover 36 that covers the test strips 20 to protect them from contamination prior to use. The holder 30 is small and can permit a user to place them in a pocket, wallet, purse, or any other desirable place to store them.

In one embodiment, the strip 20 can show the levels of bacteria using color, such as shades of color. For example, red for high levels and light pink for low levels. In another embodiment, the color may be different colors, e.g., red for high levels and blue for low levels. Other signaling techniques may also be used, other than color. For example, a section of the test strip 20 could turn grey or another color to signal the level of the reading. Multiple different colors could be used on a single strip 20 to show varied and different types of bacteria, and/or other readable features of the microbiome, if desired. There could be multiple lines of testing on the same strip 20, or separate strips 20 could be used to measure different variables.

The test strip 20 may be made of paper, plastic, cloth, or a combination thereof, or of other materials. The strip 20 may be coated with testing materials as known by those of skill in the art. The testing materials preferably remain on the strip 20 during testing and are not left on a patient's tongue or in the patient's mouth. The testing materials could be flavored, in which case, the flavoring could be left on the patient's tongue. There may be additional ways for the strip 20 to measure beyond the mouth (e.g., patients could spit into small tubes or onto strips 20 instead of swiping the mouth with a strip 20). Saliva can also be transported through breath, such that small amounts of saliva can be collected from blowing into a device with a closed mouth.

The test strips 20 can be pretreated and then show a reaction, or the reaction can be brought on when the test strips 20 (pre-treated or not) are themselves treated with a solution or the like that causes a reaction. The reaction could be a color change, for example.

Referring to FIG. 6, the invention also includes a testing system for the mouth, such as a breathalyzer 10 that tests the type and level of bacteria in the mouth. This provides a means for eliminating bad breath, and improving possible oral health issues and overall health issues. This system can be used for treating dry mouth, bad breath, gingival inflammation and other oral hygiene issues, including those associated with wearing braces.

As shown in FIG. 6, the breathalyzer 10 may include a tab 12 at the top of the breathalyzer 10 that can be inserted into the mouth. The tab 12 may serve as a sensor for holding a test strip 20. Then the breathalyzer 10 reads the bacteria level and reads out the level of bacteria on a scale, such as 1-5, where 5 is the worst. As shown in FIG. 6, the bacteria level may be presented on an electronic display 14. If the reading is at a higher level, this signals to the patient that they should use a bacteria killing product or agent.

The breathalyzer 10 has a housing 16 that includes the sensor 12. The sensor 12 includes an analyzer and a detector of diseases and disorders. In one embodiment, the sensor 12 uses a test strip 20 for capturing saliva and testing it. The sensor 12 extends outwardly from an end of the breathalyzer 10 and permits a user to put the sensor 12 in the mouth. The breathalyzer 10 may be used in home and at the office to detect oral bacteria through saliva and unbound hormones, which can provide an early indication of potential health problems that can advance the standard of care as it relates to mouth care and healthcare. Lipid imbalance is closely associated with several diseases, such as atherosclerosis, diabetes, metabolic syndromes, systemic cancers, neurodegenerative diseases, and infectious diseases. Identification of lipidomic biomarkers or key lipids in different diseases can be used to diagnose diseases and disease states. The sensor 12 can be used to test for both lipid imbalance and lipidomic biomarkers or key lipids, which can then be used for disease detection. In addition, they can be used to evaluate the response to treatments.

Biomarkers are objective, quantifiable characteristics of biological processes. The detector will initially detect bacteria that contributes to halitosis, including:

1. Gram negative bacteria located on the tongue and periodontal tissue;

2. Gram positive bacteria located on the enamel;

3. Bacteria located in saliva due to low PH or acidic saliva;

4. Bacteria production due to hydration and insufficient salivary flow; and

5. Bacteria due to food related/oral hygiene.

The sensor 12 may target 3-4 volatile organic compounds (“VOCs”) in saliva, such as ketones, alcohols, aldehyde and sulfur that are typically present in Staphylococcus aureus (S. aureus), one of the most common organisms associated with biofilms infections. Other pathogens associated with periodontitis are S. mutans, S. sanguis, P. gingivalis and F. nucleatum, which will have a different set of combination of ketones, alcohols, aldehyde, and sulfur of different compositions.

The sensor 12 and detector are meant to allow for all medical facilities and lay persons to better understand the saliva and unbound hormones as it relates to underlying diseases. Early disease detection with saliva and its abundance of biomarkers is vital to reduce disease severity and prevent complications. As discussed above, the breathalyzer 10 can use a chemically treated test strip 20, such as a cloth test strip 20, to solve for identifying the bacteria in the saliva that contributes to halitosis. The test strip 20 may have shapes other than an elongated rectangle. The test strips 20 can be easily placed into the breathalyzer 10 housing and removed therefrom. The breathalyzer 10 may alternatively include another means for capturing and testing saliva other than a test strip 20 that is removable and replaceable.

The same device can be used to detect different VOCs through the saliva and unbound hormones in the mouth. It may also be reusable, if desired.

The analyzer & detector read the type of salivary bacteria that is present in your mouth and links it to a specific disease state, on a scale differentiating different disease states. This can be done via an algorithm or computer program, if desired, and can be displayed on a computer or on an electronic display. This permits self-diagnosis similar to the scale with halitosis where you are predisposed or currently have the specific biomarkers for that disease state. This would be a tool similar to a breathalyzer 10 that you could also carry the strips 20, but it would be specific to each disease state below. This system can find 3 or 4 biomarkers for each disease state so that we are familiar with how it progresses and in what form. There can similarly be a chart to explain what the solution is based on the specific diagnosis from the lipidomic biomarker. The following disease states can be targeted; diabetes, heart disease, autoimmune disorders, oral cancers, Alzheimer's, and gastrointestinal diseases.

After using the breathalyzer 10, treatment options can be developed for use by the patient to resolve any problems identified. In addition, treatment options can be offered based upon results obtained from the test strip 20. For example, if the strip 20 turns bright red, signaling a high level of bacteria, the patient can be told to use a breath mint designed for reducing bacteria, or a mouth spray designed for reducing bacteria in the mouth. The mouth spray can be used to remove bacteria or to remove microbes, e.g., antibacterial spray or antimicrobial spray. The two features could be combined into a single spray such that one spray provides both antimicrobial and antibacterial properties. Anti-microbial treatments can be used to reduce or eliminate cavity production. Or it can be recommended that a patient use a tongue sponge.

The term “substantially,” if used herein, is a term of estimation.

While various features are presented above, it should be understood that the features may be used singly or in any combination thereof. Further, it should be understood that variations and modifications may occur to those skilled in the art to which the claimed examples pertain. The examples described herein are exemplary. The disclosure may enable those skilled in the art to make and use alternative designs having alternative elements that likewise correspond to the elements recited in the claims. The intended scope may thus include other examples that do not differ or that insubstantially differ from the literal language of the claims. The scope of the disclosure is accordingly defined as set forth in the appended claims.

What has been described above includes examples of one or more embodiments. It is, of course, not possible to describe every conceivable modification and alteration of the above devices or methodologies for purposes of describing the aforementioned aspects, but one of ordinary skill in the art can recognize that many further modifications and permutations of various aspects are possible. Accordingly, the described aspects are intended to embrace all such alterations, modifications, and variations that fall within the spirit and scope of the appended claims. Furthermore, to the extent that the term “includes” is used in either the details description or the claims, such term is intended to be inclusive in a manner similar to the term “comprising” as “comprising” is interpreted when employed as a transitional word in a claim. The term “consisting essentially,” if used herein, means the specified materials or steps and those that do not materially affect the basic and novel characteristics of the material or method. The articles “a,” “an,” and “the,” should be interpreted to mean “one or more” unless the context indicates the contrary.

What is claimed is:

1. A saliva sensing device for detecting oral health and/or overall health comprising:
a chemically treated test strip biomarker targeted to identify bacteria in the saliva.

2. The device of claim 1, wherein the test trip is targeted to analyze one or more VOCs, wherein the VOCs include one or more of ketones, alcohols, aldehyde, and sulfur.

3. The device of claim 1, wherein the test strip measures unbound hormones in the saliva, with the measured unbound hormones being tied to one or more disease states.

4. The device of claim 1, wherein the test strip measures lipidomic biomarkers.

5. The device of claim 1, wherein the test strip measures key lipids, and the measured key lipids are used in diagnosing diseases, diagnosing disease states, and developing treatment plans.

6. The device of claim 1, wherein the test strip is targeted to test pathogens including one or more of S. mutans, S. sanguis, P. gingivalis, and F. nucleatum.

7. The device of claim 1, wherein the test strip has a chemical treatment to detect gram negative bacteria.

8. The device of claim 1, wherein the test strip has a chemical treatment to detect gram positive bacteria.

9. The device of claim 1, wherein the test strip has a chemical treatment to detect one or more of the following:
1) Bacteria located in saliva due to low PH of saliva;
2) Bacteria located in saliva due to acidic saliva;
3) Bacteria production caused by insufficient hydration;
4) Bacteria production caused by insufficient salivary flow; and
5) Bacteria due to food related oral hygiene.

10. A breathalyzer for detecting bacteria in the mouth of a user comprising:
a housing;
a sensor coupled to the housing and extending outwardly therefrom for placing in the mouth of user and for collecting a saliva sample; and
an analyzer for testing saliva that contacts the sensor.

11. The breathalyzer of claim 10, wherein the sensor includes a test strip for collecting saliva.

12. The breathalyzer of claim 11, wherein the test strip is a chemically treated cloth biomarker.

13. The breathalyzer of claim 12, wherein the chemically treated cloth biomarker is configured to identify bacteria in saliva that contacts the cloth test strip.

14. The breathalyzer of claim 13, wherein the test strip detects the level of bacteria present in the saliva and the level of bacteria is determined based upon a color displayed on the test strip.

15. The breathalyzer of claim 13, wherein the test strip detects the level of bacteria present in the saliva and the level of bacteria is determined based upon a color intensity displayed on the test strip.

16. The breathalyzer of claim 12, wherein the test strip measures unbound hormones in the saliva, with the measured unbound hormones being tied to disease states.

17. The breathalyzer of claim 12, wherein the test strip measures lipidomic biomarkers

18. The breathalyzer of claim 12, wherein the test strip measures key lipids, and the measured key lipids are used in diagnosing diseases, diagnosing disease states, and developing treatment plans.

19. The breathalyzer of claim 12, wherein each test strip is targeted to a specific disease state.

20. The breathalyzer of claim 19, wherein the disease state includes one or more of diabetes, heart disease, autoimmune disorders, oral cancer, Alzheimer's, and gastrointestinal diseases.
